(19) United States
(12) Patent Application Publication
(10) Pub. No.: US 2024/0268845 A1
(43) Pub. Date: Aug. 15, 2024

(54) Piezoelectric Clot Retrieval Device

(71) Applicant: Ayman Gerges Saweres Mina, Dubai (AE)

(72) Inventor: Ayman Gerges Saweres Mina, Dubai (AE)

(21) Appl. No.: 18/434,425

(22) Filed: Feb. 6, 2024

Related U.S. Application Data
(60) Provisional application No. 63/445,790, filed on Feb. 15, 2023.

(51) Int. Cl. A61B 17/221; A61B 17/00

(52) U.S. Cl. CPC A61B 17/221; A61B 2017/00831; A61B 2017/2212

(57) ABSTRACT

A clot retrieval device has one or more segments that include a first segment and a second segment. Each of the first segment and the second segment has a generally conical body with a distal tip and a proximal mouth, the body has a lattice structure with struts crossing each other to define diamond-shaped spaces therebetween, and the body includes a piezo-electric material. A delivery wire extends though the body of the first segment and the body of the second segment, and is fixedly connected to at least the distal tip of the first segment and the distal tip of the second segment.

### BACKGROUND OF THE INVENTION

### 1. Field of the Invention

The present invention is directed to clot retrieval devices for use in removing clots from the human vasculature.

### 2. Description of the Prior Art

Current medical devices that are used for the removal of clot have limitations that reduce their effectiveness, reliability, and ease of use. For example, most of the current devices are made from super elastic Nitinol materials, and they rely primarily on the mechanical interaction between the clot retrieval element and the clot to remove the clot. As a result of the mechanical interaction, vessel damage may occur, and the efficiency of the clot removal is not ideal. Current devices are not appropriate for use in calcified, organized material because these devices function by compressing the retrieval element into the embolic material prior to attempting to remove the clot material (i.e., pure mechanical interaction). Current devices often have to embed the structure (e.g., struts) of the retrieval element into the thrombus, so this means that the retrieval element must be incorporated as part of the thrombus to remove the clot. This may result in the escape and/or breakage of the thrombus material, which can lead to the formation of distal emboli and/or new territory emboli.

Current clot retrieval devices often offer poor distal protection from secondary emboli during thrombus extraction due to the open-ended structure of the retrieval element, or loose mechanical grasping of thrombus. This may result in an intended thrombectomy procedure causing distal clot embolization and occlusion of previously patent arterial branches and collaterals. Current clot retrieval devices may be less effective when used with associated arterial stenoses due to device collapse and tendency for a stenosis to strip and debride thrombus from the clot retrieval device as the stenosis is retracted through the stenotic vessel segment.

In addition, current clot retrieval devices often require operators to choose a predetermined device length at the time of device insertion, but the chosen device length might not match the size of the target thrombus once the clot retrieval device is in the vessel and the operator is provided a closer view of the target thrombus. All above shortcomings can lead to potential vessel damage, extended procedure time, distal emboli, new territory emboli, and symptomatic or asymptomatic subarachnoid hemorrhage, among others, and hence poor clinical outcome post treatment.

Thus, there is still a need for clot retrieval devices that overcome the shortcomings outlined above.

### SUMMARY OF THE DISCLOSURE

To accomplish the objectives of the present invention, there is provided a clot retrieval device having one or more segments that include a first segment and a second segment. Each of the first segment and the second segment has a generally conical body with a distal tip and a proximal mouth, the body has a lattice structure with struts crossing each other to define diamond-shaped spaces therebetween, and the body includes a piezo-electric material. A delivery wire extends though the body of the first segment and the body of the second segment, and is fixedly connected to at least the distal tip of the first segment and the distal tip of the second segment.

The present invention overcomes the above-mentioned shortcomings through (i) the use of a new material, and (ii) a new design for the clot retrieval element. The new material is a piezoelectric material that can generate electric charges when deformed. The electric charges can improve the clot retention, and hence improve the clot removal efficiency. The new design also provides multi-segmented basket-like clot collectors that help to collect the clots, and that prevent distal and new territory emboli.

### BRIEF DESCRIPTION OF THE DRAWINGS

FIG. 1 is a perspective view of a multi-segmented clot retrieval device according to one embodiment of the present invention.

FIG. 2 is a side view of the clot retrieval device of FIG. 1.

FIG. 3 is a perspective view of one segment of the clot retrieval device of FIG. 1.

FIG. 4 is a side view of the segment of FIG. 3.

FIG. 5 is a perspective view of a multi-segmented clot retrieval device according to another embodiment of the present invention.

FIG. 6 is a side view of the clot retrieval device of FIG. 5.

FIG. 7 is a perspective view of a multi-segmented clot retrieval device according to yet another embodiment of the present invention.

FIG. 8 is a side view of the clot retrieval device of FIG. 7.

FIG. 9 is a perspective view of one segment of the clot retrieval device of FIG. 7.

FIG. 10 is a side view of the segment of FIG. 9.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

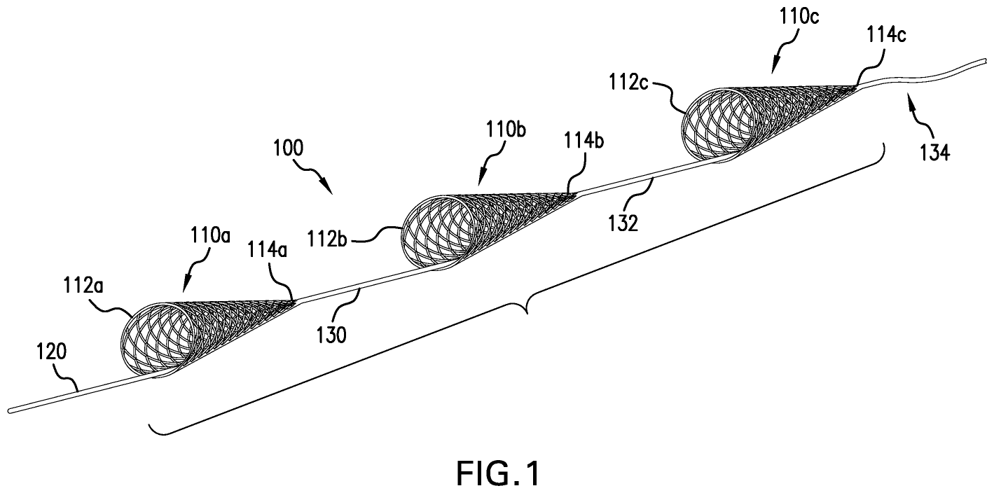

FIGS. 1-4 illustrate a clot retrieval device 100 according to one embodiment of the present invention. The clot retrieval device 100 has a plurality of segments 110a, 110b and 110c. Segment 110a can be considered to be a first-stage clot collector, segment 110b can be considered to be a second-stage clot collector, and segment 110c can be considered to be a third-stage clot collector. A single delivery wire 120 is fixedly connected to a part of the proximal mouth 112a of the segment 110a, and extends along and through the body 116a of the segment 110a. The delivery wire 120 extends through the delivery catheter or sheath (not shown) and is connected to a control handle (not shown). A first connector 130 (which is part of the single delivery wire 120) fixedly connects the distal tip 114a of the segment 110a to a part of the proximal mouth 112b of the segment 110b, and the delivery wire 120 extends along and through the body 116b of the segment 110b. A second connector 132 (which is part of the single delivery wire 120) fixedly connects the distal tip 114b of the segment 110b to a part of the proximal mouth 112c of the segment 110c, and the delivery wire 120 extends along and through the body 116c of the segment 110c. A soft atraumatic distal tip 134 extends from the distal tip 114c of the segment 110c.

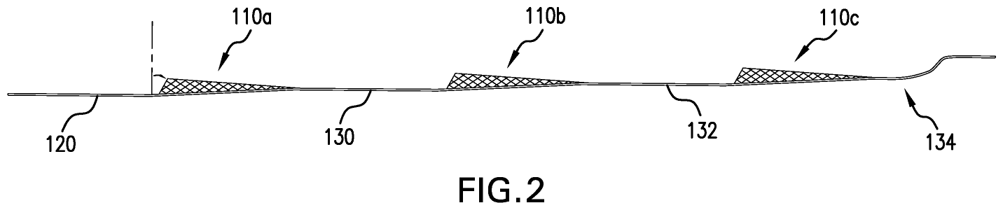

Even though three segments 100a-100c are shown in FIGS. 1-2, it is possible to for the clot retrieval device 100 to one, two or more segments, and up to twenty segments, depending on the location of the clot and the relevant clinical needs.

Each segment 110a, 110b and 110c can assume two profiles, an expanded configuration for use in clot collection when inside a blood vessel, and a compressed configuration when the clot retrieval device 100 is inside a delivery catheter.

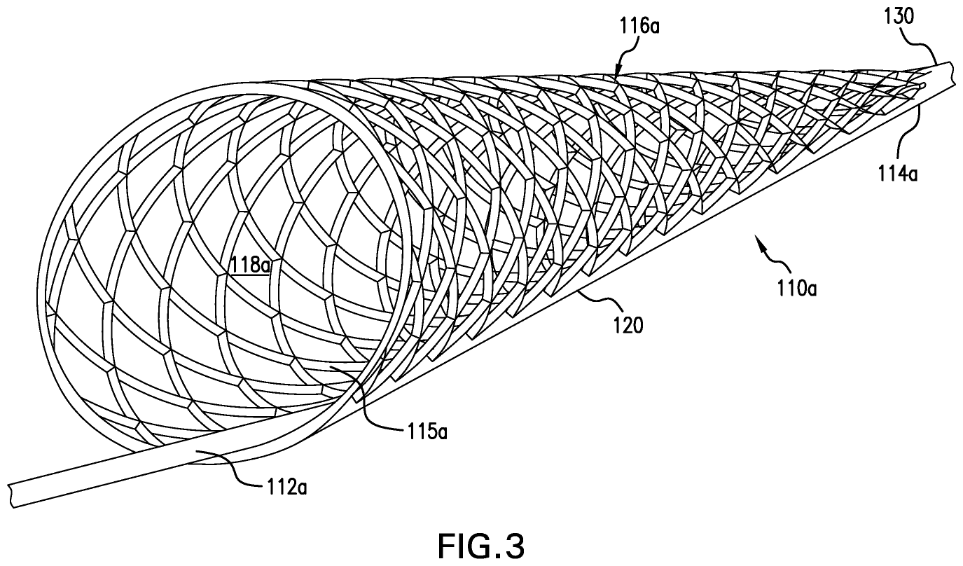

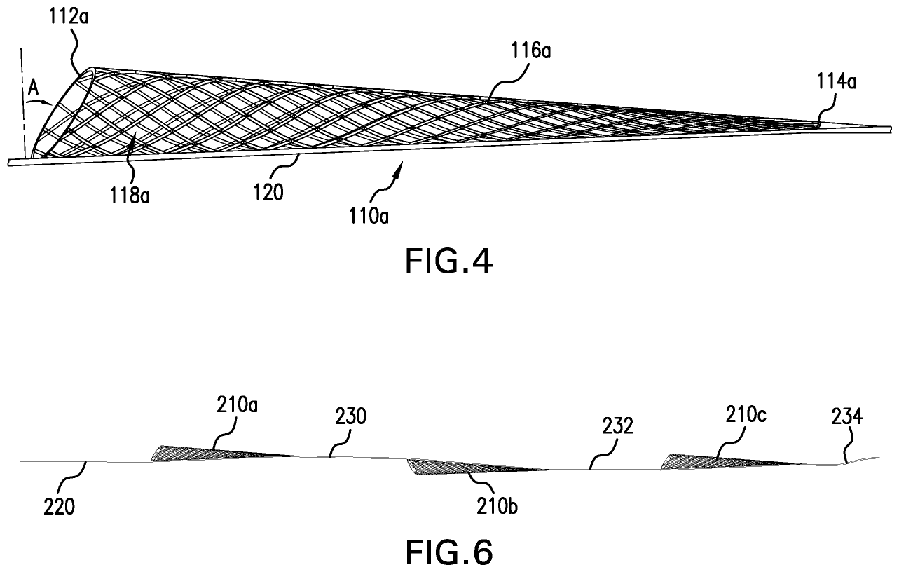

FIGS. 3-4 provide an enlarged view of the segment 110a. The other segments 110b and 110c can be identical to the segment 110a or have different diameters. The segment 110a has a generally conical body 116a with a distal tip 114a and a proximal mouth 112a. The proximal mouth 114a is angled by a radial angle A which can range from 1 to 90 degrees. The diameter of the body 116a is greatest at the proximal mouth 112a and tapers towards the distal tip 114a, which is essentially a tip with the smallest diameter and connects to the distal end of the connector 130.

The body 116a has a lattice structure with struts 115a crossing each other to define diamond-shaped spaces 118a therebetween. The sizes of the spaces 118a can vary from one end to another end. For example, the sizes can be larger closer to the proximal mouth 112a and smaller closer to the distal tip 114a. The body 116a is preferably made from a wire-braided structure to ensure maximum deformity or flexibility. The body 116a can be made from a biocompatible polymer, nickel-titanium (Nitinol), a cobalt-chromium alloy, or other appropriate metallic alloys.

The delivery wire 120 can be secured (e.g., by welding) at the proximal mouth 114a and at the distal tip 114a, and extend through the lattice structure (i.e., in and out of the spaces 118a) of the body 116a.

The length of the segment 110a can range from 3 mm to 70 mm, the height of the segment 110a can range from 1 mm to 50 mm from the distal tip 114a to the proximal mouth 112a (which has the greatest height). The length of the connectors 130 and 132 can range from 1 mm to 50 mm.

Radiopaque markers (not shown) can be provided at any location on the proximal mouth 112a, along the body 116a, at the distal tip 114a or along any of the connectors 130 and 132. In addition, a surface modification or coating can be provided on the surface of the body 116a to provide certain functionalites, such as lubricity, more effective clot retention, and to minimize trauma. Examples can include lubricious coatings, drugs, biological materials and others.

An important part of the present invention is that the material for the body 116a includes piezo-electric materials. Piezo-electric materials are materials that have piezo-electricity. Piezoelectricity is the effect of mechanical strain/deformation and electric fields/potential on a material. Mechanical strain/deformation on piezoelectric materials will produce a polarity/potential in the material, and applying an electric field/potential to a piezoelectric material will create strain/deformation/bend within the material. When pressure is applied to a piezoelectric material, a dipole and net polarization/potential are produced in the direction of the applied stress/deformation. Piezoelectricity converts different types of energy into another type of energy. In the present invention, the conversion between the mechanical energy/deformation/bend to electricity/potential is used.

The piezo-electric materials can be either piezo-electric ceramic and some ferroelectric materials (for example BaTiO₃, etc.), or piezo-electric polymer materials (for example, polyvinylidene fluoride, etc.), or the combination of the different types of piezo-electric materials (composite materials). The piezo-electric materials can be in the form of layers, sheet, tubular, thin film, surface layer, deposited or plated layer, printed layer, powder form, fibers, wires, textile/fabric, strip form, electrospun fibers, electrospun nanofibers, etc.

The entire segment 110a can be made from piezoelectric materials, or the piezoelectric materials can be integrated into conventional materials (e.g., nickel-titanium) to provide the piezoelectric properties.

In use, the when the segment 110a is deformed, stressed, strained or bent, electric charge can be generated on the surface of the body 116a to help with clot retention. Specifically, piezoelectric materials can generate electrons by exposing it to deformity (e.g., deployment of the body 112a inside the clot) so that this electric current (flow of electrons) creates electrostatic charges opposite to platelet charges so that the body 116a attracts the platelet-rich thrombus more than compared to a conventional clot retrieval body that does not have piezoelectric material. The electrical charges can be generated either by applying external (AC/DC) electricity via the delivery wire 120, or via the deployment of the clot retrieval device 100 through a delivery catheter, where the deformity experienced through the deployment would generate electrical charges. In this regard, the delivery wire 120 can be made of stainless-steel, Nitinol or cobalt chromium, so as to allow transmission of electrical energy therethrough. In addition, the piezoelectric materials can be provided in the form of a diode or dielectric membrane to concentrate the charges or generating more electrostatic potential compared to the thrombus/blood Zeta potential.

Each segment 110a, 110b and 110c preferably has the same construction, but the different segments 110a, 110b and 110c can have different sizes (e.g., diameters). For example, the most distal segment 110c can have the smallest diameter/length and the most proximal segment 110a can have the largest diameter/length.

The segments 110a, 110b and 110c form basket-like clot collectors. The multi-stage clot collection offered by this configuration allows each stage to act as an independent filter, and as mentioned above, all the filters could be of the same size or the sizes can be gradually adjusted from the distal tip 114a to the proximal mouth 112a. Each segment 110a, 110b and 110c should have the maximum deformity during usage.

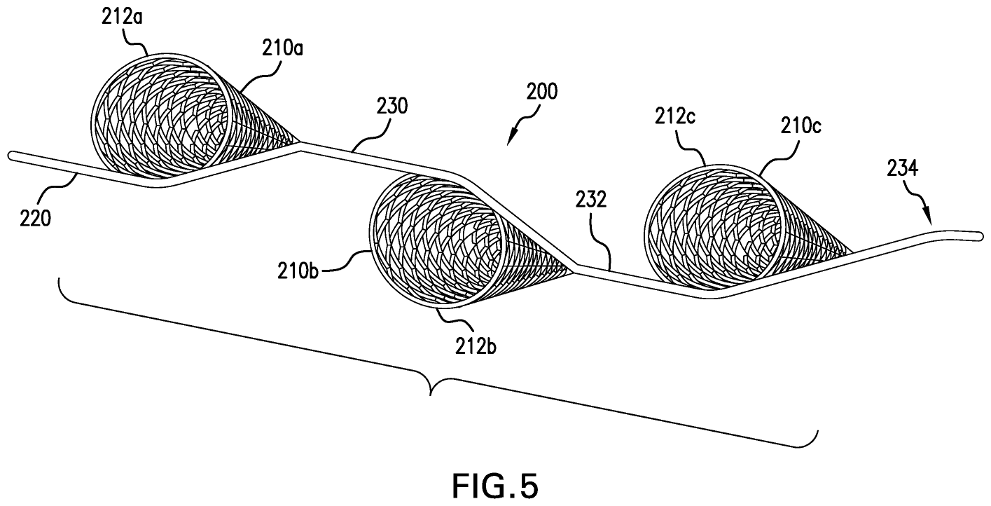

FIGS. 5-6 illustrate a clot retrieval device 200 according to another embodiment of the present invention. The clot retrieval device 200 has a plurality of segments 210a, 210b and 210c connected by connectors 230 and 232, with each segment 210a, 210b, 210c being identical to the segments 110a, 110b, 110c, and the connectors 230 and 232 being the same as connectors 130 and 132. The only difference between the two clot retrieval devices 100 and 200 is the locations of the connectors and the delivery wire 120 and 220. In the clot retrieval device 100, the single delivery wire 120 (and its connectors 130, 132 and distal tip 234) are connected to the proximal mouths 112a, 112b and 112c, respectively, along the same longitudinal line. In the clot retrieval device 200, the single delivery wire 220 and its connectors 230, 232 are connected to the proximal mouths 212a, 212b and 212c, respectively, along alternating longitudinal lines. For example, the delivery wire 220 and connector 232 are connected to the proximal mouths 212a and 212c, respectively, along the same longitudinal line, but the connector 230 is connected to the proximal mouth 212b along a longitudinal line (defined by the connector 230) that is offset by 180 degrees from the longitudinal line defined by the delivery wire 220 and connector 232.

The difference between the orientations of the connectors in the clot retrieval devices 100 and 200 is related to the anatomy and minimizing damage to blood vessels. When retrieving the clot retrieval device 100 or 200 with the thrombus inside a retrieval (delivery) catheter, the force applied at the curvature of the blood vessel is high and can stretch and possibly perforate the blood vessel. By having differently-oriented segments 100a-100c, it is possible to (i) minimize central wire bias and tension, and (ii) minimize the force by the deflection of the segments 100a-100c during clot retrieval.

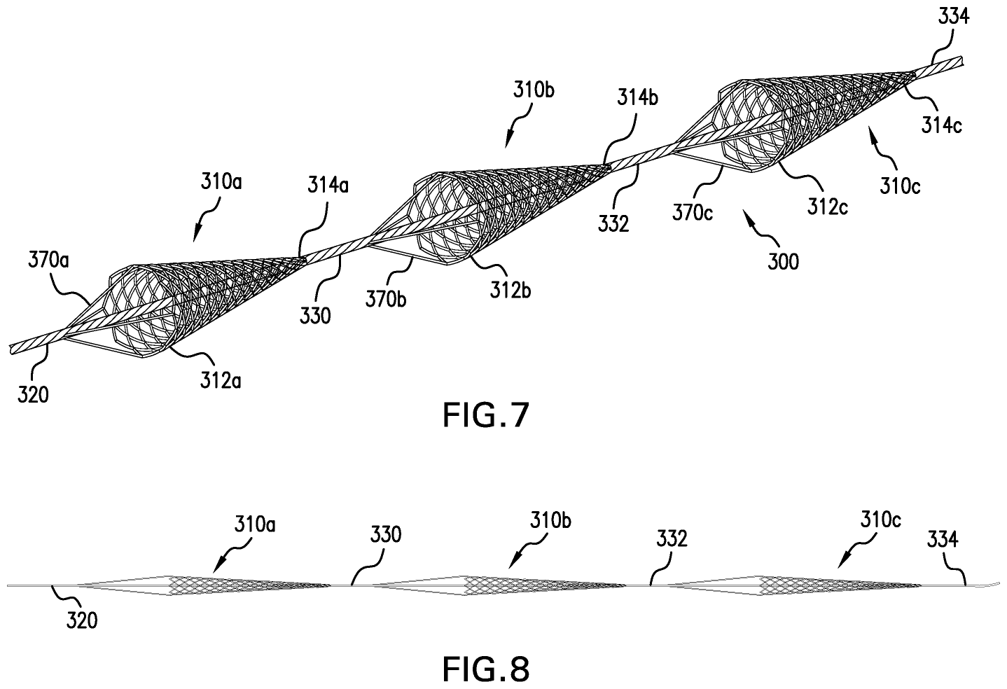

FIGS. 7-10 illustrate a clot retrieval device 300 according to a third embodiment of the present invention. The clot retrieval device 300 has a plurality of segments 310a, 310b and 310c. A single delivery wire 320 extends through the longitudinal center of each segment 310a, 310b and 310, connecting the distal tips 314a, 314b and 314c of the three segments 310a, 310b and 301c. The delivery wire 320 extends through the delivery catheter or sheath (not shown) and is connected to a control handle (not shown). A first connector 330 fixedly connects the distal tip 314a of the segment 310a to the distal tip 314b, and a second connector 332 fixedly connects the distal tip 314b of the segment 310b to the distal tip 314c of the segment 310c. A soft atraumatic distal tip 334, which is part of the delivery wire 320, extends from the distal tip 314c. Even though three segments 300a-300c are shown in FIGS. 7-8, it is possible for the clot retrieval device 300 to be provided with one, two or more segments, and up to twenty segments, depending on the location of the clot and the relevant clinical needs.

Each segment 310a, 310b and 310c can assume two profiles, an expanded configuration for use in clot collection when inside a blood vessel, and a compressed configuration when the clot retrieval device 300 is inside a delivery catheter.

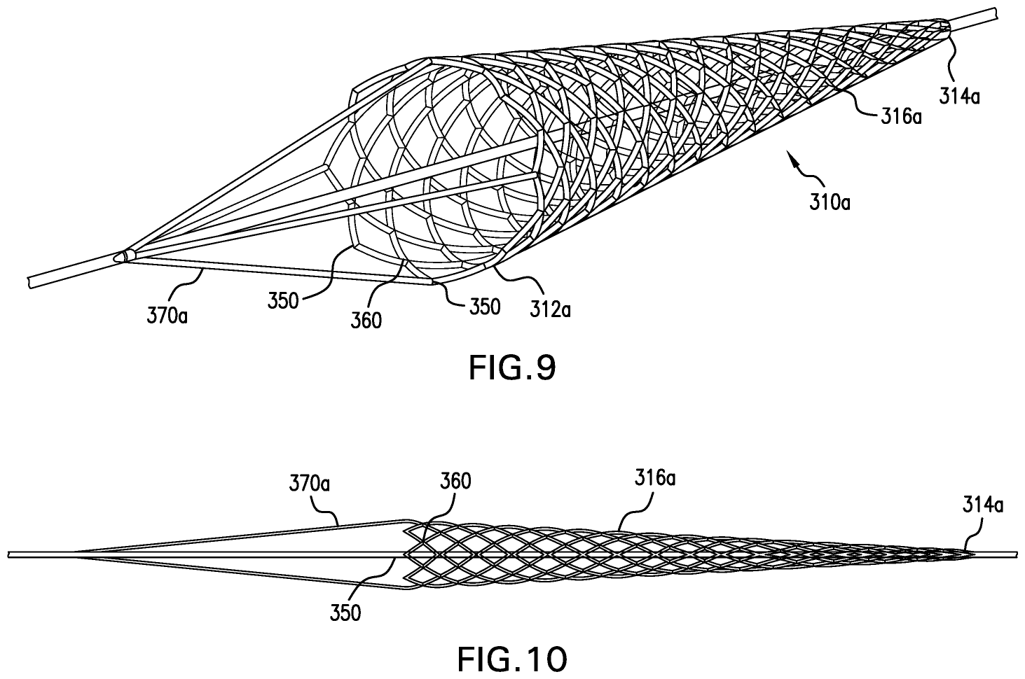

FIGS. 9-10 provide an enlarged view of the segment 310a. The other segments 310b and 310c are identical to segment 310a. The segment 310a also has a generally conical body 316a with a distal tip 314a and a proximal mouth 312a. The diameter of the body 316a is greatest at the proximal mouth 312a and tapers towards the distal tip 314a, which is essentially a tip with the smallest diameter and connects to the distal end of the connector 330.

The segment 310a is essentially the same as the segment 110a except that the proximal mouth 314a is not angled, and the proximal mouth 314a has alternating peaks 350 and valleys 360. A plurality of connecting wires 370a are attached to selected peaks 350 at a first proximal end thereof, with a second distal end is connected to the delivery wire 320. Four connecting wires 370a are shown in FIGS. 7-10, but any number of connecting wires 370a can be provided. The segments 310b and 310c each has a plurality of connecting wires 370b and 370c, respectively, which have distal ends connected to the connectors 330 and 332, respectively.

Each segment 110a-110c, 210a-210c and 310a-310c represents a separate filter. The clot retrieval devices 100 and 200 have eccentric filters (segments) and the clot retrieval device 300 has concentric filters (segments). This eccentric vs. concentric distinction is related to technical requirements and different applications can use different embodiments depending on the clinical needs. Specifically, the delivery wire 120, 220, 330 needs to be connected to the distal end of each segment because thrombus is supposedly retained inside each segment and the segments need to be supported or connected at the distal ends so that the thrombus retained therein can be pulled out. Eccentric segments 100a-100c, 200a-200c have a lower profile but require a high delivery and retrieval force. In contrast, concentric segments 300a-300c have a larger profile (i.e., requires a larger catheter lumen) but low delivery and retrieval force.

As a result, the structure and arrangement of the filters (segments) in the clot retrieval devices 100, 200 and 300 provides for (i) a small entry profile when the filters are compressed inside the delivery catheter, and (ii) strong deformity when the filters are deployed inside the thrombus to generate charges.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A clot retrieval device, comprising:
a first segment and a second segment, each of the first segment and the second segment having a generally conical body with a distal tip and a proximal mouth, the body has a lattice structure with struts crossing each other to define diamond-shaped spaces therebetween, and the body includes a piezo-electric material; and
a delivery wire extending though the body of the first segment and the body of the second segment, and fixedly connected to at least the distal tip of the first segment and the distal tip of the second segment.

2. The device of claim 1, wherein the body of the first segment and the second segment is composed partially of piezo-electric material.

3. The device of claim 1, wherein the body of the first segment and the second segment is completely made of piezo-electric material.

4. The device of claim 1, wherein the piezo-electric material can be either piezo-electric ceramic, a ferroelectric material, piezo-electric polymer materials, or a combination of the different types of piezo-electric materials.

5. The device of claim 1, wherein the piezo-electric material is provided in the form a deposited layer, a sheet, tubular, a thin film, a surface layer, a plated layer, a printed layer, powder form, fibers, wires, fabric, strip form, electrospun fibers, or electrospun nanofibers.

6. The device of claim 1, wherein the delivery wire is fixedly connected to the proximal mouth of the first segment, and includes a connector that fixedly connects the distal tip of the first segment and the proximal mouth of the second segment.

7. The device of claim 6, wherein the delivery wire is provided along a longitudinal line that is the same as a longitudinal line defined by the connector.

8. The device of claim 6, wherein the delivery wire is provided along a longitudinal line that is offset by 180 degrees from a longitudinal line defined by the connector.

9. The device of claim 1, wherein the piezo-electric element provides a source of electric energy.

10. The device of claim 1, wherein the proximal mouth of the first segment is angled by a radial angle.

11. The device of claim 1, wherein the body of the first segment and the second segment is greatest at the proximal mouth and tapers towards the distal tip.

12. The device of claim 7, wherein the connector is a first connector, further including a third segment having a generally conical body with a distal tip and a proximal mouth, the body of the third segment has a lattice structure with struts crossing each other to define diamond-shaped spaces therebetween, and the body of the third segment including a piezo-electric material;
wherein the delivery wire also extends though the body of the third segment and is fixedly connected to the distal tip of the third, the delivery wire further including a second connector fixedly connecting the distal tip of the second segment and the proximal mouth of the third segment; and
wherein the second connector is provided along a longitudinal line that is the same as a longitudinal line defined by the first connector.

13. The device of claim 8, wherein the connector is a first connector, further including a third segment having a generally conical body with a distal tip and a proximal mouth, the body of the third segment has a lattice structure with struts crossing each other to define diamond-shaped spaces therebetween, and the body of the third segment including a piezo-electric material;
wherein the delivery wire also extends though the body of the third segment and is fixedly connected to the distal tip of the third, the delivery wire further including a second connector fixedly connecting the distal tip of the second segment and the proximal mouth of the third segment; and
wherein the second connector is provided along a longitudinal line that is the same as the longitudinal line defined by the delivery wire.

14. The device of claim 1, wherein a first plurality of connecting wires connect the delivery wire to the proximal mouth of the first segment, and a second plurality of connecting wires connect the connector to the proximal mouth of the second segment.

15. The device of claim 14, wherein the proximal mouth of the first segment is defined by alternating peaks and valleys, and wherein a proximal end of each of the first plurality of connecting wires is connected to a separate one of the peaks.

16. A clot retrieval device, comprising a segment having a generally conical body with a distal tip and a proximal mouth, the body has a lattice structure with struts crossing each other to define diamond-shaped spaces therebetween, and the body includes a piezo-electric material, and a delivery wire extending though the body of the segment and fixedly connected to at least the distal tip of the segment.

17. The device of claim 16, wherein the piezo-electric element provides a source of electric energy.

18. The device of claim 16, wherein the proximal mouth is angled by a radial angle.

19. The device of claim 16, wherein the piezo-electric material can be either piezo-electric ceramic, a ferroelectric material, piezo-electric polymer materials, or a combination of the different types of piezo-electric materials.

20. The device of claim 16, wherein the piezo-electric material is provided in the form a deposited layer, a sheet, tubular, a thin film, a surface layer, a plated layer, a printed layer, powder form, fibers, wires, fabric, strip form, electrospun fibers, or electrospun nanofibers.
